(19) United States
(12) Patent Application Publication
(10) Pub. No.: US 2010/0248335 A1
(43) Pub. Date: Sep. 30, 2010

(54) METHOD AND APPARATUS FOR TREATMENT OF ORGANIC WASTE

(76) Inventors: Toshihiro Komatsu, Hyogo (JP); Tomoko Matsuzaki, Hyogo (JP); Shin-ichiro Wakahara, Hyogo (JP)

Correspondence Address: KUSNER & JAFFE;HIGHLAND PLACE SUITE 310, 6151 WILSON MILLS ROAD, HIGHLAND HEIGHTS, OH, 44143, US

(21) Appl. No.: 12/733,755

(22) PCT Filed: Sep. 22, 2008
(86) PCT No.: PCT/JP2008/002607

§ 371 Date: Mar. 18, 2010

(30) Foreign Application Priority Data
Sep. 25, 2007 (JP) 2007 247420

(51) Int. Cl. B09B 3/00; C12M 1/12

(52) U.S. Cl. 435/262.5; 435/283.1

(57) ABSTRACT

The pH condition in a methane fermentation tank is adjusted in a predetermined range equal to or less than the intended pH condition causing no scale formation, while being maintained in a predetermined range approximate to an optimized pH condition suitable for methane fermentation, and thereby the formation of inorganic scales and the like is suppressed.

### TECHNICAL FIELD

The present invention relates to a method and an apparatus for treatment of organic waste, and concerns a technique for suppressing the formation of inorganic scales and the like in the membrane methane fermentation treatment of organic waste containing a large amount of phosphorus, magnesium, and the like.

### BACKGROUND ART

In recent years, biomass ethanol has received attention as an energy source, in terms of renewability and carbon neutrality. Biomass ethanol is ethanol produced by fermenting biomass, such as sugarcane and corn and distilling, which is thus fermented ethanol or brewed ethanol. By-products produced from the process for manufacturing this ethanol include carbon dioxide, fermentation residues, stillage, and the like, and the treatment of these is problematic.

Much of the stillage of bioethanol contains a large amount of phosphorus, magnesium, and the like because the bioethanol raw material components are concentrated. Examples of the method for treatment of organic waste containing a large amount of phosphorus, magnesium, and the like include a treatment method described in Japanese Patent Laid-Open No. 2003-275726 (hereinafter referred to as Patent Document 1).

In this treatment method, an iron-based flocculant is added to organic waste, the organic waste is subjected to methane fermentation treatment under anaerobic conditions, the fermented liquid is aerated to oxidize Fe²⁺ in the fermented liquid to Fe³⁺, and then, the fermented liquid is separated into the liquid and dehydrated sludge.

In this treatment method, the production of magnesium ammonium phosphate and hydrogen sulfide, which are inhibitory factors for the methane fermentation treatment, is suppressed by adding the iron-based flocculant to the organic waste and subjecting the organic waste to methane fermentation treatment under anaerobic conditions. Further, the alkalinity of the fermented liquid is decreased by reducing the iron component of the iron-based flocculant under anaerobic conditions and consuming part of the reduced component for the fixation of phosphorus and sulfur contained in the organic waste, and a basic nitrogen compound produced with the methane fermentation treatment.

### DISCLOSURE OF THE INVENTION

### Problems to be Solved by the Invention

When organic waste containing a larger amount of phosphorus, magnesium, and nitrogen, such as the stillage for bioethanol described above, than common organic waste, is subjected to methane fermentation treatment, magnesium ammonium phosphate (MAP) is produced as inorganic scales and plugs the piping and the like.

Particularly, when organic waste containing a large amount of phosphorus, magnesium, and nitrogen is subjected to membrane methane fermentation treatment, the formed inorganic scales are fixed to the membrane surface as well as the piping, to cause separation membrane malfunction, and as the fermentation inhibiting substances are not sufficiently discharged, high-concentration methane fermentation treatment through the concentration of the digestion liquid cannot be carried out. Thus, the inorganic scales are a factor inhibiting methane fermentation treatment.

Also, in methane fermentation treatment, pH is important as a fermentation condition, and the optimum methane fermentation condition is pH=about 8. Therefore, when a large amount of an agent, such as an iron-based flocculant, is added to the methane fermentation tank to suppress the formation of scales, as in Patent Document 1, the amount of the agent added, such as an iron-based flocculant, increases as the amount of phosphorus, magnesium, and nitrogen becomes larger. Therefore, it is difficult to maintain the optimum methane fermentation conditions.

The present invention solves the foregoing problems. It is an object of the present invention to provide a method and an apparatus for treatment of organic waste that suppress the formation of inorganic scales and the like in the membrane methane fermentation treatment of organic waste containing a large amount of phosphorus, magnesium, and nitrogen.

### SUMMARY OF THE INVENTION

In order to solve the foregoing problems, a method for treatment of organic waste according to the present invention includes circulating a digestion liquid between a methane fermentation tank having raw material organic waste containing an inorganic scale forming substance flowing thereinto and a membrane separation tank having a membrane separation apparatus submerged therein; and adding a pH adjusting agent to the digestion liquid in the membrane separation tank or the digestion liquid flowing from the methane fermentation tank into the membrane separation tank, thereby adjusting the pH condition in the membrane separation tank in a predetermined range equal to or less than the intended pH condition causing no scale formation, while maintaining the pH condition in the methane fermentation tank in a predetermined range approximate to an optimized pH condition suitable for methane fermentation.

By the above-described configuration, in membrane methane fermentation treatment using the methane fermentation tank and the membrane separation tank separate from each other, in the methane fermentation tank, the nitrogen component contained in the raw material is decomposed under anaerobic conditions by the action of the methane bacteria in the tank, and ammonium nitrogen is produced with the generation of biogas.

Therefore, in the methane fermentation tank in normal operation, the pH of the digestion liquid in the tank increases, and the digestion liquid in the tank under the progress of methane fermentation has pH=about 8. It is required for the optimized methane fermentation to maintain the pH condition in the methane fermentation tank in a predetermined range approximate to an optimized pH condition=8.

On the other hand, in the membrane separation tank, no fluctuations in pH occur, and the pH of the digestion liquid in the membrane separation tank is about 8, equal to the pH of the digestion liquid in the methane fermentation tank. This is because the digestion liquid that has underwent sufficient fermentation in the methane fermentation tank, that is, the digestion liquid in the final stage of fermentation and decomposition, flows into the membrane separation tank.

However, when the raw material contains a large amount of nitrogen, phosphorus, magnesium, and the like, the precipitation of inorganic crystals of magnesium ammonium phosphate (MAP) and the like becomes more significant as the pH condition of the digestion liquid in the membrane separation tank increases due to the relationship between pH and a solubility product. These inorganic scales of MAP and the like are a factor fouling the membrane surfaces of the membrane separation apparatus.

Therefore, the pH of the digestion liquid in the membrane separation tank is adjusted low, using the pH adjusting agent, and the pH condition is controlled at the intended pH condition in which the solubility product of the MAP and the like is higher, that is, crystals do not precipitate easily, for example, a pH condition is lower than 7.8, whereby the formation of the inorganic scales is suppressed, and the membrane surfaces of the membrane separation apparatus can be kept sound.

As the pH adjusting agent, inorganic acids, such as hydrochloric acid and sulfuric acid, or organic acids, such as citric acid and acetic acid, are used, or iron-based flocculants being acids, such as polyferric sulfate and ferric chloride, and simultaneously having dephosphorization and desulfurization effect, magnesium chloride being acidic in a solution, and the like are used.

Alternatively, a CO₂-rich gas with increased CO₂ concentration is removed from biogas generated in the methane fermentation tank, by means of a CO₂ concentrator, and this CO₂-rich gas is blown into the membrane separation tank to dissolve the carbonic acid gas in the digestion liquid, and thus the pH condition in the membrane separation tank is controlled to be lower than the intended pH condition, thereby avoiding troubles in the membrane separation tank.

The digestion liquid in the membrane separation tank adjusted to pH<7.8 is returned to the methane fermentation tank again by the above-described operation. For the circulation level of this digestion liquid, the flow rate is very low, compared with the amount of the liquid in the methane fermentation tank. As one example, with respect to the amount of the liquid in the methane fermentation tank, 5.0 Qm³, the circulation level of the liquid between the methane fermentation tank and the membrane separation tank is 5.0 Qm³/d. This is such that the digestion liquid in the methane fermentation tank is replaced once per day.

Therefore, the level of the digestion liquid returned from the membrane separation tank to the methane fermentation tank has little effect on fluctuations in pH in the methane fermentation tank during normal operation, and the digestion liquid in the methane fermentation tank is maintained at pH=about 8.

As a result, the pH condition in the membrane separation tank is controlled at the intended pH condition, whereby the pH condition in the methane fermentation tank is maintained at the optimized pH condition. And the optimization of the methane fermentation in the methane fermentation tank, and the suppression of the formation of MAP and the like in the membrane separation tank can be simultaneously achieved.

When the raw material contains a large amount of nitrogen, phosphorus, magnesium, and the like, and it is difficult to maintain the intended pH condition in the membrane separation tank, and maintain the optimized pH condition in the methane fermentation tank, the digestion liquid is concentrated by a concentration and separation unit during the transfer of the digestion liquid from the methane fermentation tank to the membrane separation tank to separate the digestion liquid into concentrated sludge containing the inorganic scale forming substance as inorganic sludge and into a separated liquid. And the concentrated sludge is discharged therefrom as surplus digestion sludge, and thereby, the inorganic scale forming substances in the digestion liquid supplied to the membrane separation tank are decreased. Also, a pressure-driven membrane separation apparatus, such as an internal or external pressure-driven one, can be used instead of the membrane separation tank having the membrane separation apparatus submerged therein.

### ADVANTAGES OF THE INVENTION

As described above, according to the present invention, in the membrane methane fermentation treatment of organic waste containing a large amount of phosphorus, magnesium, and ammonia, the pH condition in the membrane separation tank is controlled at the intended pH condition, whereby the pH condition in the methane fermentation tank is maintained at the optimized pH condition, and the optimization of the methane fermentation in the methane fermentation tank, and the suppression of the formation of the inorganic scales in the membrane separation tank can be simultaneously achieved.

Therefore, the operability of the membrane separation apparatus is excellent. At the same time, the attachment of the inorganic scales to the membrane surfaces can be suppressed with a small amount of the agent used, and the methane fermentation can be optimized without decreasing fermentation efficiency.

### BRIEF DESCRIPTION OF THE DRAWINGS

FIG. 1 is a schematic diagram showing an apparatus for treatment of organic waste in an embodiment of the present invention;

FIG. 2 is a schematic diagram showing an apparatus for treatment of organic waste in another embodiment of the present invention;

FIG. 3 is a schematic diagram showing an apparatus for treatment of organic waste in another embodiment of the present invention;

FIG. 4 is a schematic diagram showing an apparatus for treatment of organic waste in another embodiment of the present invention;

FIG. 5 is a graph diagram showing changes in the pH of a membrane-permeated liquid over time; and

FIG. 6 is a graph diagram showing changes in membrane permeation flux over time.

### BEST MODE FOR CARRYING OUT THE INVENTION

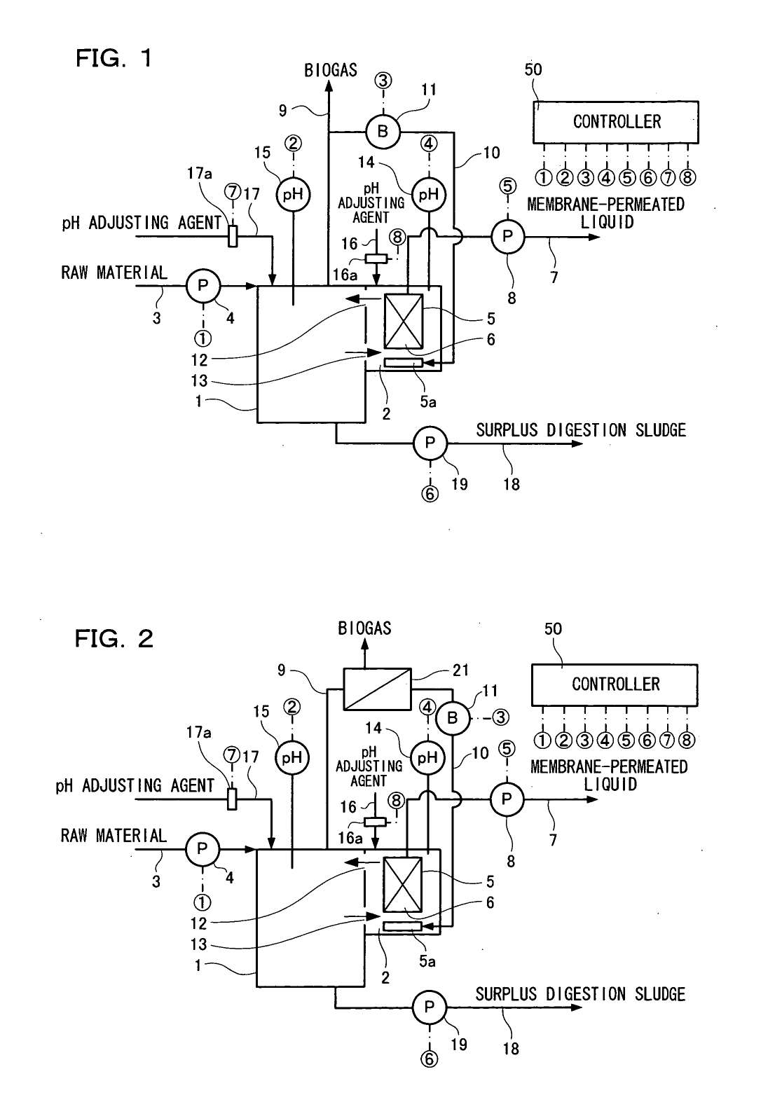

The embodiments of the present invention will be described below, based on the drawings. In FIG. 1, an apparatus for treatment of organic waste according to this embodiment performs membrane methane fermentation treatment, and a methane fermentation tank 1 and a membrane separation tank 2 are separate. The methane fermentation tank 1 is connected to a raw material supply system 3, and organic waste as raw material flows into the methane fermentation tank 1, as driven by a pump 4.

A membrane separation apparatus 5 is submerged in the membrane separation tank 2. Various forms, such as hollow fiber membranes, tubular membranes, and flat-sheet membranes, can be applied to the membrane separation apparatus 5. Here, the membrane separation apparatus 5 includes a plurality of flat-sheet membrane cartridges 6 and a diffuser 5a for emitting biogas as a membrane surface cleaning gas from under the flat-sheet membrane cartridges 6. In the membrane cartridge 6, a filtration membrane is located on both surfaces of a filter plate. The membrane separation apparatus 5 is in communication with a membrane-permeated liquid suction system 7 and operated at suction pressure applied by a suction pump 8 as driving pressure.

The methane fermentation tank 1 is of an enclosed type, and a biogas discharge system 9 is connected to the tank top part. In the biogas discharge system 9, a branch pipe 10 is connected to the diffuser 5a of the membrane separation apparatus 5 via a blower 11.

In this embodiment, the methane fermentation tank 1 and the membrane separation tank 2 are in communication via an overflow path 12 and an underflow path 13, and a digestion liquid is circulated between the methane fermentation tank 1 and the membrane separation tank 2. However, it is also possible to locate the methane fermentation tank 1 and the membrane separation tank 2 at distant positions and connect the methane fermentation tank 1 and the membrane separation tank 2 by a circulation system including piping. pH meters 14 and 15 for measuring the pH of the digestion liquid in the tanks are respectively provided in the membrane separation tank 2 and the methane fermentation tank 1. The pH meter 14 provided in the membrane separation tank 2 is a requirement in the present invention.

pH adjusting agent supply systems 16 and 17 as pH adjusting agent adding units are respectively connected to the membrane separation tank 2 and the methane fermentation tank 1. The pH adjusting agent supply system 16 connected to the membrane separation tank 2 is a requirement in the present invention. The pH adjusting agent supply systems 16 and 17 include flow rate adjusting units 16a and 17a, such as pumps and valves, and add a suitable amount of a pH adjusting agent to the digestion liquid in the tanks.

Inorganic acids, such as hydrochloric acid and sulfuric acid, organic acids, such as citric acid and acetic acid, iron-based flocculants being acids, such as polyferric sulfate and ferric chloride, and simultaneously having dephosphorization and desulfurization effect, magnesium chloride being acidic in a solution, and the like are used as the pH adjusting agent.

The pH adjusting agent supply system 17 is necessary only when the pH condition in the membrane separation tank cannot be made equal to the intended pH condition only by the pH adjusting agent supply system 16, or when the pH condition in the methane fermentation tank 1 is out of a predetermined range. Not only acid but also an alkali substance, such as NaOH, may be supplied.

When the methane fermentation tank 1 and the membrane separation tank 2 are connected by a circulation system including piping, the pH adjusting agent supply system 16 provided in the membrane separation tank 2 can also be provided so as to add the pH adjusting agent to the digestion liquid flowing from the methane fermentation tank 1 into the membrane separation tank 2.

In the pH adjusting agent supply systems 16 and 17, the amount of the pH adjusting agent added can also be adjusted by manual operation, but the amount is adjusted by a controller 50 in this embodiment.

The controller 50 controls the units, such as the above-described pumps and blower, and controls the amount of the pH adjusting agent added by the pH adjusting agent supply systems 16 and 17, using the measured values of the pH meters 14 and 15 as indicators. A surplus digestion sludge discharge system 18 is in communication with the lower part of the methane fermentation tank 1, and the digestion liquid in the tank removed as driven by a discharge pump 19 is transferred therefrom as surplus digestion sludge.

The operation in the above-described configuration will be described below. The organic waste as raw material is quantitatively supplied from the raw material supply system 3 to the methane fermentation tank 1 as driven by the pump 4. In the methane fermentation tank 1, the raw material is decomposed by methane bacteria in the tank, and the digestion liquid is circulated between the methane fermentation tank 1 and the membrane separation tank 2.

In the membrane separation tank 2, the digestion liquid is solid-liquid separated by the membrane separation apparatus 5, the membrane-permeated liquid is removed by the suction pump 8 therefrom through the membrane-permeated liquid suction system 7 therefrom, and the concentrated liquid is returned to the methane fermentation tank 1. Biogas generated in the methane fermentation tank 1 is discharged therefrom through the biogas discharge system 9, and part of the biogas is supplied to the diffuser 5a of the membrane separation apparatus 5 through the branch pipe 10 as driven by the blower 11. An upward flow caused by the biogas diffused in the membrane separation tank 2 flows in a solid-gas-liquid multiphase flow along the membrane surfaces of the flat-sheet membrane cartridges 6 and cleans the membrane surfaces.

In the methane fermentation tank 1, ammonium nitrogen is produced with the generation of biogas. Therefore, in the methane fermentation tank 1 in normal operation, the pH of the digestion liquid in the tank increases, and the digestion liquid in the tank under the progress of methane fermentation has pH=about 8. In order to optimize the methane fermentation, it is required to maintain the pH condition in the methane fermentation tank in a predetermined range approximate to an optimized pH condition=8.

On the other hand, in the membrane separation tank 2, no fluctuations in pH occur, and the pH of the digestion liquid in the membrane separation tank is about 8, equal to the pH of the digestion liquid in the methane fermentation tank. This is because the digestion liquid that has underwent sufficient fermentation in the methane fermentation tank 1, that is, the digestion liquid in which the progress of fermentation and decomposition has nearly finished, flows into the membrane separation tank 2.

However, when the organic waste, the raw material, contains a large amount of nitrogen, phosphorus, magnesium, and the like, the precipitation of inorganic crystals of magnesium ammonium phosphate (MAP) and the like becomes more significant as the pH condition of the digestion liquid in the membrane separation tank increases due to the relationship between pH and a solubility product. These inorganic scales of MAP and the like are a factor fouling the membrane surfaces of the flat-sheet membrane cartridges 6 of the membrane separation apparatus 5.

Therefore, the controller 50 provides a suitable amount of the pH adjusting agent to the digestion liquid in the membrane separation tank 2, using the measured value of the pH meter 14 provided in the membrane separation tank 2 as an indicator, and adjusts the pH condition in the membrane separation tank to the intended pH condition causing no inorganic scale formation, here, in a predetermined range of pH<7.8 or less (6.8<pH<7.8), by the adjustment of the amount of the pH adjusting agent added. The intended pH condition can also be in a predetermined range of pH<8 or less (6.6<pH<8). Also in this adjustment, the pH condition in the methane fermentation tank is maintained in a predetermined range approximate to the optimized pH condition (pH=8) suitable for methane fermentation (the condition of this range is that the methane fermentation is maintained well).

In other words, the pH of the digestion liquid in the membrane separation tank is adjusted low, using the pH adjusting agent, whereby the pH condition is controlled at the intended pH condition (pH<7.8) in which the solubility product of the MAP and the like is high, that is, crystals do not precipitate easily, to suppress the formation of the inorganic scales, and thereby, the membrane surfaces of the membrane separation apparatus 2 can be maintained sound.

The digestion liquid in the membrane separation tank adjusted to pH<7.8 is returned to the methane fermentation tank 1 again by the above-described operation. For the circulation level of this digestion liquid, the flow rate is very low, compared with the amount of the liquid in the methane fermentation tank. In this embodiment, with respect to the amount of the liquid in the methane fermentation tank 1, 5.0 Qm³, the circulation level of the liquid between the methane fermentation tank 1 and the membrane separation tank 2 is set to 5.0 Qm³/d. This is such that the digestion liquid in the methane fermentation tank is replaced once per day.

Therefore, the amount of the digestion liquid returned from the membrane separation tank 2 to the methane fermentation tank 1 has little effect on fluctuations in pH in the methane fermentation tank during normal operation, and the digestion liquid in the methane fermentation tank 1 is maintained in the predetermined range approximate to the optimized pH condition (pH=8).

In this manner, in this embodiment, the pH condition in the membrane separation tank is controlled at the intended pH condition, whereby the optimization of the methane fermentation in the methane fermentation tank 1, and the suppression of the formation of the inorganic scales of MAP and the like in the membrane separation tank 2 can be simultaneously achieved.

Also, the controller 50 monitors the pH of the digestion liquid in the methane fermentation tank 1 by the pH meter 15 provided in the methane fermentation tank 1. When the pH of the digestion liquid in the methane fermentation tank 1 deviates from the predetermined range approximate to the optimized pH condition (pH=8), the controller 50 adds a suitable amount of the pH adjusting agent to the digestion liquid in the membrane separation tank 2 from the pH adjusting agent supply system 17, using the measured value of the pH meter 15 as an indicator, and maintains the optimization of the methane fermentation. In this manner, the pH conditions in the membrane separation tank 2 and the methane fermentation tank 1 can also be individually controlled using both the pH adjusting agent supply system 17.

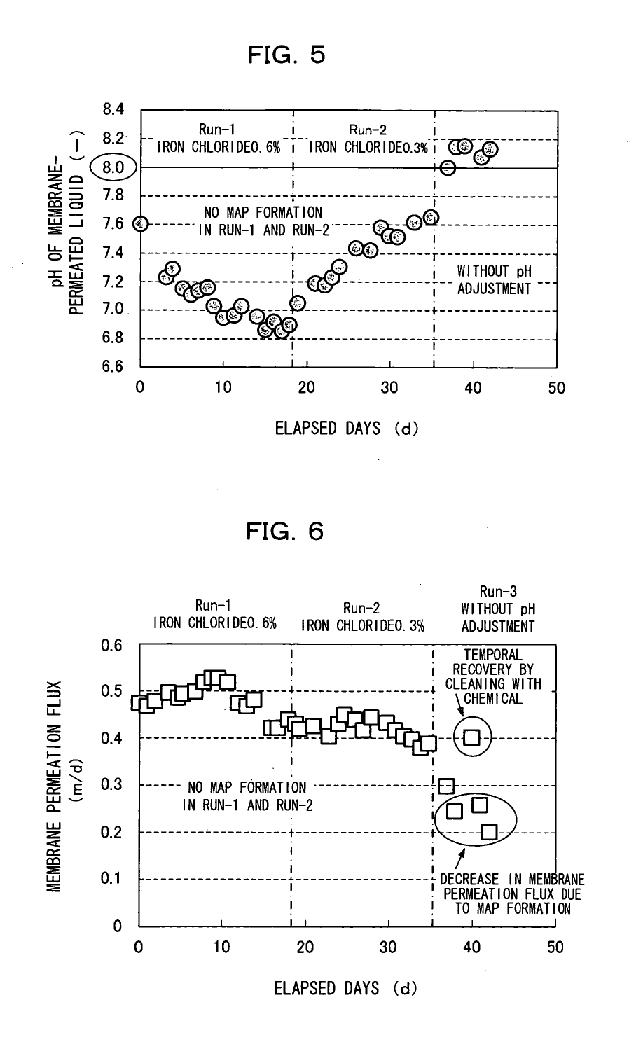

FIG. 5 and FIG. 6 show the experimental data of pH in the membrane separation tank (without adjustment or adjusted by the addition of iron chloride), and decrease in the membrane permeation flux of the membrane separation apparatus due to MAP crystal formation, when bioethanol stillage is subjected to the above-described membrane methane fermentation treatment. As shown in FIG. 5, in Run 1 with the addition of 0.6% ferric chloride, and Run 2 with the addition of 0.3% ferric chloride, the pH of the membrane-permeated liquid is maintained at 7.8 or less, and no MAP is formed. However, in Run 3 without pH adjustment, the pH is 8 or more. Also, as shown in FIG. 6, in Run 1 with the addition of 0.6% ferric chloride, and Run 2 with the addition of 0.3% ferric chloride, the membrane permeation flux is maintained at substantially more than 0.4 m/d, and no MAP is formed. However, in Run 3 without pH adjustment, the membrane permeation flux decreases to 0.3 m/d or less, and is temporarily recovered by cleaning with a chemical, but the membrane permeation flux immediately decreases to 0.3 m/d or less.

The pH value in the methane fermentation tank during the periods of Run 1, Run 2, and Run 3 was maintained near pH=8 without the addition of the pH adjusting agent to the methane fermentation tank.

FIG. 2 shows another embodiment of the present invention. The same numerals denote constituent members similar to those in the previous embodiment, and description is omitted. In a configuration shown in FIG. 2, a CO₂ concentrator 21 is provided as a CO₂ concentrating unit. The primary side of the CO₂ concentrator 21 is in communication with a biogas discharge system 9, and the secondary side is in communication with a branch pipe 10. The CO₂ concentrator 21 separates a CO₂-rich gas with increased CO₂ concentration from biogas generated in a methane fermentation tank 1. The CO₂-rich gas is supplied to the diffuser 5a of a membrane separation apparatus 5 and blown into a digestion liquid to dissolve the carbonic acid gas in the digestion liquid. In this embodiment, the CO₂-rich gas is diffused in the digestion liquid in a membrane separation tank, but can also be blown into the digestion liquid flowing from the methane fermentation tank 1 into the membrane separation tank 2.

The controller 50 controls a blower 11, using the measured value of the pH meter 14 of the membrane separation tank 2 as an indicator, to adjust the amount of the CO₂-rich gas blown by the CO₂ concentrator 21. The pH condition in the membrane separation tank is adjusted in a predetermined range equal to or less than the intended pH condition (pH<7.8) causing no scale formation by the adjustment of the amount of the CO₂-rich gas by this controller, and thereby the pH condition in the methane fermentation tank is maintained in a predetermined range approximate to an optimized pH condition (pH=8) suitable for methane fermentation.

In this manner, in this embodiment, the CO₂-rich gas is blown into the membrane separation tank 2 to dissolve the carbonic acid gas in the digestion liquid, and thereby, the pH condition in the membrane separation tank is controlled to be lower than the intended pH condition, avoiding troubles in the membrane separation tank 2. It is also possible to accessorily add a suitable amount of a pH adjusting agent from pH adjusting agent supply systems 16 and 17, based on the measured values of the pH meter 14 and a pH meter 15, as in the previous embodiment, as required.

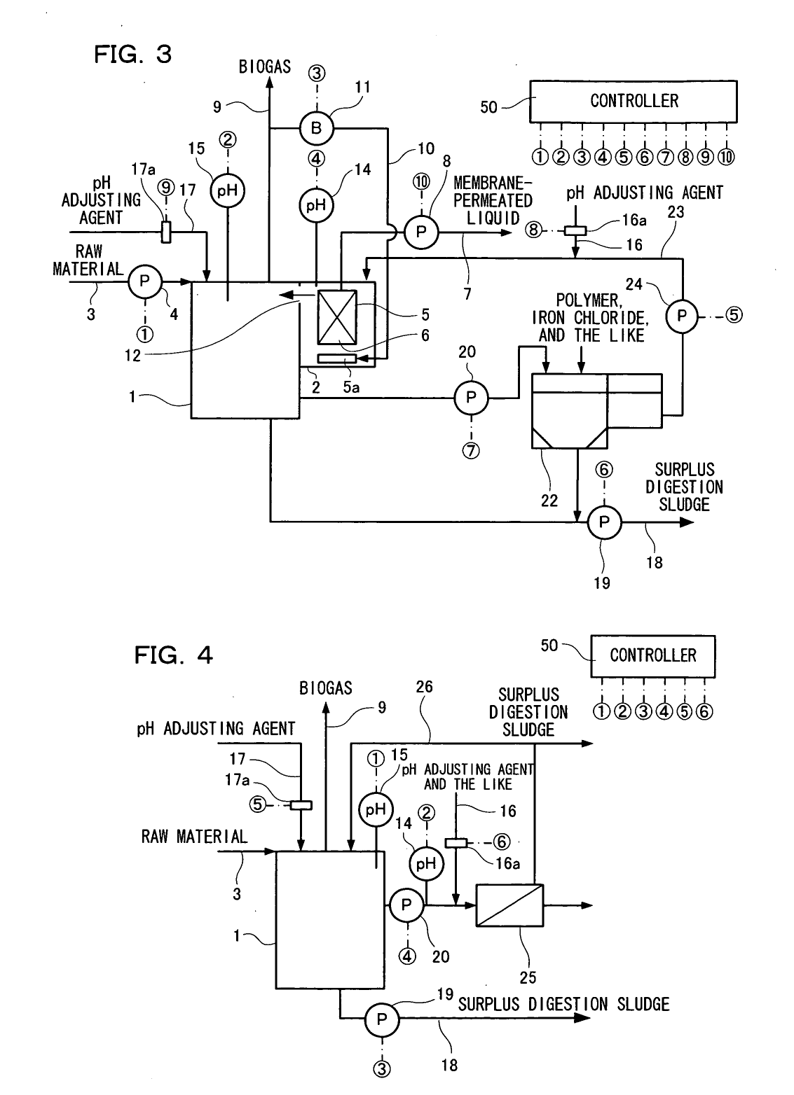

FIG. 3 shows another embodiment of the present invention. The same numerals denote constituent members similar to those in the previous embodiments, and description is omitted. In a configuration shown in FIG. 3, basically, treatment related to methane fermentation, as in the previous embodiments, is performed, but a case is assumed where the raw material contains a large amount of phosphorus, magnesium, nitrogen, and the like, whereby it is difficult to maintain the pH condition in a membrane separation tank at the intended pH condition, and maintain the pH condition in a methane fermentation tank at an optimized pH condition.

Therefore, a concentration and separation tank 22 is provided as a concentration and separation unit. In this embodiment, the concentration and separation tank 22 is a coagulating sedimentation tank, but a mechanical centrifugal concentrator, a multidisk type, and the like can also be used as the concentration and separation unit.

The inflow side of the concentration and separation tank 22 is connected to a methane fermentation tank 1 via a feed pump 20, and the outflow side is in communication with a membrane separation tank 2 via a return system 23.

By this configuration, in the concentration and separation tank 22, an iron-based flocculant having dephosphorization and desulfurization effect is added to a digestion liquid in the methane fermentation tank 1 supplied by the feed pump 20 to separate the digestion liquid into concentrated sludge and a separated liquid. The concentrated sludge contains inorganic scale forming substances, such as phosphorus, magnesium, and ammonia, as inorganic sludge, and this concentrated sludge is discharged therefrom as surplus digestion sludge, and thereby, the inorganic scale forming substances in the digestion liquid supplied to the concentration and separation tank 22 are decreased. The separated liquid is transferred, with the inorganic scale forming substances decreased, to the membrane separation tank 2 via the return system 23 by a pump 24.

FIG. 4 shows another embodiment of the present invention. The same numerals denote constituent members similar to those in the previous embodiments, and description is omitted. In a configuration shown in FIG. 4, basically, treatment related to methane fermentation, as in the previous embodiments, is performed, but a pressure type membrane separation apparatus 25 is used as a membrane separation apparatus.

The primary side of the pressure type membrane separation apparatus 25 is in communication with a methane fermentation tank 1 via a feed pump 20, and the secondary side is in communication with methane fermentation 1 via a sludge return system 26. A pH adjusting agent supply system 16 adds a pH adjusting agent to a digestion liquid supplied from the methane fermentation tank 1 to the pressure type membrane separation apparatus 25. A pH meter 14 measures the pH of the digestion liquid supplied from the methane fermentation tank 1 to the pressure type membrane separation apparatus 25.

By this configuration, the digestion liquid transferred from the methane fermentation tank 1 is concentrated and separated into a separated liquid and concentrated sludge by the pressure type membrane separation apparatus 25. The separated liquid is removed therefrom, and the concentrated sludge is returned to the methane fermentation tank 1 through the sludge return system 26.

The controller 50 controls the amount of the pH adjusting agent added by the pH adjusting agent supply system 16, using the measured value of the pH meter 14 as an indicator, thereby adjusting the pH condition in the pressure type membrane separation apparatus in a predetermined range equal to or less than the intended pH condition causing no scale formation, while maintaining the pH condition in the methane fermentation tank in a predetermined range approximate to an optimized pH condition suitable for methane fermentation.

It is also possible to provide together the concentration and separation tank 22 as the concentration and separation unit in the previous embodiment in a case where the raw material contains a large amount of nitrogen, phosphorus, magnesium, nitrogen, and the like, whereby it is difficult to maintain the pH condition in the membrane separation tank at the intended pH condition, and maintain the pH condition in the methane fermentation tank at the optimized pH condition.

1. A method for treatment of organic waste, comprising: circulating a digestion liquid between a methane fermentation tank having raw material organic waste containing an inorganic scale forming substance flowing thereinto and a membrane separation tank having a membrane separation apparatus submerged therein; and
adding a pH adjusting agent to the digestion liquid in the membrane separation tank or to the digestion liquid flowing from the methane fermentation tank into the membrane separation tank,
thereby adjusting a pH condition in the membrane separation tank in a predetermined range equal to or less than an intended pH condition causing no scale formation, while maintaining a pH condition in the methane fermentation tank in a predetermined range approximate to an optimized pH condition suitable for methane fermentation.

2. A method for treatment of organic waste, comprising: circulating a digestion liquid between a methane fermentation tank having raw material organic waste containing an inorganic scale forming substance flowing thereinto and a membrane separation tank having a membrane separation apparatus submerged therein;
separating a CO₂-rich gas from biogas generated in the methane fermentation tank; and
blowing the CO₂-rich gas into the digestion liquid in the membrane separation tank or into the digestion liquid flowing from the methane fermentation tank into the membrane separation tank to dissolve CO₂,
thereby adjusting a pH condition in the membrane separation tank in a predetermined range equal to or less than an intended pH condition causing no scale formation, while maintaining a pH condition in the methane fermentation tank in a predetermined range approximate to an optimized pH condition suitable for methane fermentation.

3. The method for treatment of organic waste according to claim 1 or 2, comprising:
concentrating the digestion liquid drawn from the methane fermentation tank, by a concentration and separation unit, to separate the digestion liquid into a concentrated sludge containing the inorganic scale forming substance as inorganic sludge and a separated liquid;
discharging the concentrated sludge therefrom as surplus digestion sludge; and
transferring the separated liquid to the membrane separation tank.

4. A method for treatment of organic waste, comprising: drawing a digestion liquid from a methane fermentation tank having raw material organic waste containing an inorganic scale forming substance flowing thereinto;
concentrating the digestion liquid by a pressure type membrane separation apparatus to separate the digestion liquid into a separated liquid and a concentrated sludge;
drawing the separated liquid in the pressure type membrane separation apparatus therefrom;
returning the concentrated sludge in the pressure type membrane separation apparatus to the methane fermentation tank; and
adding a pH adjusting agent to the digestion liquid supplied from the methane fermentation tank to the pressure type membrane separation apparatus,
thereby adjusting a pH condition in the pressure type membrane separation apparatus in a predetermined range equal to or less than an intended pH condition causing no scale formation, while maintaining a pH condition in the methane fermentation tank in a predetermined range approximate to an optimized pH condition suitable for methane fermentation.

5. The method for treatment of organic waste according to any one of claims 1, 2 or 4, wherein the organic waste contains phosphorus, magnesium, and nitrogen as the inorganic scale forming substances, and the intended pH condition is adjusted to 6.6<pH<8.0, preferably 6.8<pH<7.8.

6. An apparatus for treatment of organic waste, comprising:
a methane fermentation tank having raw material organic waste containing an inorganic scale forming substance flowing thereinto;
a membrane separation tank having a digestion liquid circulated between the methane fermentation tank and the membrane separation tank;
a membrane separation apparatus submerged in the membrane separation tank;
a pH adjusting agent adding unit for adding a pH adjusting agent to the digestion liquid in the membrane separation tank or to the digestion liquid flowing from the methane fermentation tank into the membrane separation tank;
a pH meter for measuring a pH of the digestion liquid in the membrane separation tank; and
a controller for controlling an amount of the pH adjusting agent added by the pH adjusting agent adding unit, using a measured value of the pH meter,
wherein by adjustment of the amount of the pH adjusting agent by the controller, a pH condition in the membrane separation tank is adjusted in a predetermined range equal to or less than an intended pH condition causing no scale formation, while a pH condition in the methane fermentation tank is maintained in a predetermined range approximate to an optimized pH condition suitable for methane fermentation.

7. An apparatus for treatment of organic waste, comprising:
a methane fermentation tank having raw material organic waste containing an inorganic scale forming substance flowing thereinto;
a membrane separation tank having a digestion liquid circulated between the methane fermentation tank and the membrane separation tank;
a membrane separation apparatus submerged in the membrane separation tank;
a CO₂ concentrating unit for separating a CO₂-rich gas from biogas generated in the methane fermentation tank and blowing the CO₂-rich gas into the digestion liquid in the membrane separation tank or into the digestion liquid flowing from the methane fermentation tank into the membrane separation tank;
a pH meter for measuring a pH of the digestion liquid in the membrane separation tank; and
a controller for controlling an amount of the CO₂-rich gas blown by the CO₂ concentrating unit, using a measured value of the pH meter,
wherein by adjustment of the amount of the CO₂-rich gas by the controller, a pH condition in the membrane separation tank is adjusted in a predetermined range equal to or less than an intended pH condition causing no scale formation, while a pH condition in the methane fermentation tank is maintained in a predetermined range approximate to an optimized pH condition suitable for methane fermentation.

8. The apparatus for treatment of organic waste according to claims 6 or 7, comprising a concentration and separation unit for concentrating the digestion liquid transferred from the methane fermentation tank to separate the digestion liquid into concentrated sludge containing the inorganic scale forming substance as inorganic sludge and a separated liquid, discharging the concentrated sludge therefrom as surplus digestion sludge, and transferring the separated liquid to the membrane separation tank.

9. An apparatus for treatment of organic waste, comprising:
a methane fermentation tank having raw material organic waste containing an inorganic scale forming substance flowing thereinto;
a pressure type membrane separation apparatus for concentrating a digestion liquid transferred from the methane fermentation tank to separate the digestion liquid into a separated liquid and concentrated sludge, removing the separated liquid therefrom, and returning the concentrated sludge to the methane fermentation tank;
a pH adjusting agent adding unit for adding a pH adjusting agent to the digestion liquid supplied from the methane fermentation tank to the pressure type membrane separation apparatus;
a pH meter for measuring a pH of the digestion liquid supplied from the methane fermentation tank to the pressure type membrane separation apparatus; and
a controller for controlling an amount of the pH adjusting agent added by the pH adjusting agent adding unit, using a measured value of the pH meter,
wherein by adjustment of the amount of the pH adjusting agent by the controller, a pH condition in the pressure type membrane separation apparatus is adjusted in a predetermined range equal to or less than an intended pH condition causing no scale formation, while a pH condition in the methane fermentation tank is maintained in a predetermined range approximate to an optimized pH condition suitable for methane fermentation.
